(19) United States
(12) Patent Application Publication
(10) Pub. No.: US 2010/0280123 A1
(43) Pub. Date: Nov. 4, 2010

(54) FOOD SUPPLEMENT CONTAINING ALPHA-KETO ACIDS FOR SUPPORTING DIABETES THERAPY

(75) Inventors: Andreas KARAU, Vieux Moulin (FR); Henrike Gebhardt, Münster (DE); Norbert Windhab, Hofheim (DE); Matthias Kottenhahn, Freigericht Somborn (DE); Yuefei Liu, Neu-Ulm (DE); Jürgen M. Steinacker, Ulm (DE)

Correspondence Address: SMITH, GAMBRELL & RUSSELL, 1130 CONNECTICUT AVENUE, N.W., SUITE 1130, WASHINGTON, DC, 20036, US

(73) Assignee: Evonik Degussa GmbH, Essen (DE)

(21) Appl. No.: 12/752,706

(22) Filed: Apr. 1, 2010

(30) Foreign Application Priority Data
Apr. 3, 2009 (DE) 102009016119.8

(51) Int. Cl. A61K 31/194; A61P 3/10; A61P 5/50; A61K 31/19; A23L 1/30; A23L 1/302; A23D 9/00; A23L 1/304; A23L 1/305

(52) U.S. Cl. 514/574; 514/557; 426/648; 426/72; 426/541; 426/601; 426/74

(57) ABSTRACT

Food supplement containing alpha-keto acids for supporting diabetes therapy. The present invention relates to a formulation which is used as food supplement and contains alpha-keto acids for supporting therapy in diabetes mellitus type II (DM).

The present invention relates to a formulation which is used as food supplement and contains alpha-keto acids for supporting therapy in diabetes mellitus, in particular of type II.

Numerous studies show that the incidence of type II DM can be lowered by physical training. Physical training is the best preventive measure and is at the same time also one of the most important therapeutic possibilities for treatment of DM. It has been demonstrated that physical training leads to an improvement of glucose metabolism and thereby also of the clinical course.

Physical training leads to muscular adaptation in which a number of cellular processes take place, which include, inter alia, muscle damage, muscle regeneration, muscle hypertrophy and also muscle fibre transformation. In these cellular processes, energy and protein metabolism plays a critical role. Amino acids are important participants in this case.

With diabetics, however, carrying out physical training is made more difficult in that they suffer from muscle atrophy. One of the causes of muscle atrophy is that, because of reduced availability of glucose for energy production, proteins can be broken down for energy production.

Alpha-keto acids have differing functions in metabolism. The keto acid analogues of branched-chain amino acids play an important role in amino acid metabolism, especially in skeletal muscle and in the liver. One third of muscle protein consists of the branched-chain amino acids which cannot be formed by the body, but must be taken in with the diet. In the muscle, particularly in the case of physical exertion, proteins are continuously synthesized and broken down, wherein during breakdown of an amino acid the corresponding alpha-keto acid is formed by transferring the amino group to a carrier. The resultant keto acid can then be further oxidized enzymatically for energy production. The carrier is transported to the liver and there liberates ammonia which is converted into urea and excreted via the kidneys.

The use of alpha-keto acids, which are derived from branched-chain amino acids, for nutritive purposes has long been known. For instance, in particular alpha-ketoisocaproate (ketoleucine) can be used for reducing protein breakdown in muscle and for a reduction of the urea formation, which results from the protein breakdown, after muscle operations (U.S. Pat. No. 4,677,121). The use of ketoleucine in undernourishment, muscular dystrophy or uraemia, or in other disorders which result as secondary consequences of protein breakdown in the muscle, is also described there. Ketoleucine is administered intravenously in this case.

In the functional food sector, the branched-chain amino acids, especially, are used directly for supporting muscle synthesis, e.g. in the case of athletes (Shimomura, Y. et al., American Society for Nutrition). However, it is known that the increased nitrogen supply via the amino acids leads to an increased liberation of ammonia in the muscle, which in turn leads to fatigue symptoms.

The use of alpha-keto acids for improving muscle performance and for supporting muscle recuperation after stress is described in U.S. Pat. No. 6,100,287, wherein salts of the corresponding anionic keto acids with cationic amino acids as counterion such as arginine or lysine, for example, are used. However, polyamines are also formed thereby, of which it is known that they can lead to apoptosis (programmed cell death). Also, the breakdown products of polyamines are excreted by the kidneys which are increasingly stressed thereby. An intake of arginine or lysine is therefore not advisable.

There is a need for food supplements which, in the case of diabetics, in particular having diabetes mellitus type II, promote the feeling of wellbeing and the efficiency during and after sporting activities, and furthermore help a diabetic metabolic state to normalize.

The problem is solved by providing a formulation which contains at least one of the alpha-keto acids of the group alpha-keto-isocaproate (KIC), alpha-ketoisovalerate (KIV), alpha-keto-beta-methylvalerate (KMV) and alpha-ketoglutarate (AKG), is essentially nitrogen-free and preferably does not contain any nitrogenous compounds. The formulation is a food supplement and optionally contains in addition further vitamins and minerals.

Essentially nitrogen-free means that the nitrogen content of the formulation is less than 6% by weight, preferably less than 3% by weight, in particular less than 0.5% by weight, based on the total weight.

In addition to the alpha-keto acids, salts thereof can also be present in the formulation according to the invention. Suitable salts are in particular the alkali metal or alkaline earth metal salts, in particular the Na⁺, K⁺, Ca²⁺ and Mg²⁺ salts of the said alpha-keto acids.

A preferred embodiment is formulations which comprise a combination of alpha-ketoglutarate and alpha-ketoisocaproate, or alpha-ketoglutarate and alpha-ketoisovalerate, or alpha-keto-glutarate and alpha-keto-beta-methylvalerate, or a combination of all four alpha-keto acids and/or salts thereof. Preferably, a quantitative ratio of AKG to BCKA (branched-chain keto acids) in the formulation of 5:1 to 1:5 is established, in particular 3:1 to 1:3, preferably 2:1 to 1:2. The daily dose of the alpha-keto acids taken up via the formulation should not exceed the amount of 2000 mg/kg of body weight. Preference is given to doses of between 10 mg/kg and 1000 mg/kg of body weight for AKG and 10 mg/kg and 1000 mg/kg for the BCKAs. Particularly preferred doses are in the range from 25 mg/kg to 150 mg/kg of body weight for AKG, KIC, KIV and KMV, with the proviso that this gives in the case of adults an approximate total amount of alpha-keto acid taken in of 1.25 g to 25 g.

Furthermore, other additives can be added to the formulation. Those which may be emphasized in particular are compounds which promote the regeneration process such as, for example, vitamins, in particular vitamin A, vitamin B₁, B₂, B₆ and B₁₂, vitamin C, vitamin D, vitamin E, vitamin K, pantothenic acid, niacin, folic acid, biotin, choline and inositol. In addition, antioxidants such as, for example beta-carotene, potassium citrate, citric acid, lactic acid, tocopherol, sodium or potassium ascorbate, or ascorbic acid, can be present in the formulation. Minerals and trace elements from the group sodium, potassium, magnesium, calcium, iron, zinc, manganese, copper, selenium, chromium, phosphorus and iodine are likewise possible as additions. The said additives are added in this case in the amounts conventional for the food sector.

A formulation is taken to mean a product which is active in the field which is technically relevant here with the participation of the person, and has a defined and reproducible composition with respect to individual substances/substance groups of interest, with which the body is intended to be supplied in a targeted manner with one or more specific substances. Of course, this encompasses the fact that the substance in question has an exact dose in a formulation. Formulations are correspondingly administered in a dosage form, in the form of capsules, tablets or the like.

Preferably, formulations can contain, for example (the quantities represent the respective preferred daily dose):

10-500 mg of sodium,
10-500 mg of potassium,
50-500 mg of calcium,
10-300 mg of magnesium,
1-20 mg of zinc,
5-50 mg of iron,
0.1-1 mg of iodine,
5-100 μg of selenium,
5-100 lag of chromium,
up to 100 mg of vitamin B₁,
up to 100 mg of vitamin B₂,
up to 100 mg of vitamin B₆,
up to 200 μg of vitamin B₁₂,
up to 5 g of vitamin C,
up to 500 mg of vitamin E,
up to 300 mg of pantothenic acid,
up to 1 g of niacin,
up to 10 mg of folic acid,
up to 1 mg of biotin.

Further additives which come into consideration as an addition are saturated or unsaturated fatty acids, in particular C₆-C₂₂-fatty acids. Use can be made of, for example, fatty acids of fats and oils from the group sunflower oil, sesame oil, rapeseed oil, palm oil, castor oil, coconut oil, safflower oil, soya oil, pig lard and beef tallow. In addition, preservatives, food dyes, sweeteners, taste enhancers and/or flavourings can be present in the food supplement in the customary amounts known to those skilled in the art. If the additives employed are used in relatively large amounts, recourse is made to nitrogen-free additives. Particularly preferred food supplements do not contain any nitrogenous additives.

The claimed formulations can be used, for example in the form of a powder, a tablet or in the form of a solution or suspension. In tablet form, the alpha-keto acids or salts thereof are preferably formulated with approximately 30 to 90 percent by volume in the formulation, preferably using nitrogen-free additives, in particular poorly absorbable carbohydrates and fats (oils), and optionally amino acids are present, in particular L-ornithine or L-arginine, wherein the amounts are set in the ranges of the stated nitrogen contents of the total amount of the preparation.

If direct administration of the formulations in the form of a powder or a tablet is desired, the addition of customary carriers can be advantageous. Suitable carriers are, for example linear or (hyper)branched polyesters, polyethers, polyglycerols, polyglycolides, polylactides, polylactide-co-glycolides, polytartrates and polysaccharides, or poly(ethylene oxide)-based dendrimers, polyether dendrimers, coated PAMAM dendrimers such as, for example, polylactide-co-glycolide coating, or polyarylethers.

The powder or the tablets can in addition be provided with a covering in order, for example, to enable release of the food supplement only in the intestinal tract. The following capsule casing materials are preferably used in this case: carboxy-methylcellulose, nitrocellulose, poly(vinyl alcohol), shellac, carrageenan, alginates, gelatin, cellulose acetate, phthalates, ethylcellulose, polyglycerols, polyesters or Eudragit®.

If, in contrast, the formulation is administered in the form of a solution or suspension of the food supplement, addition of emulsifiers or colloids can be useful in order to be able to take up all desired components as well as possible in an aqueous solution. Suitable additions are, e.g., poly(vinyl alcohol)s, glycerides of edible fatty acids, esters thereof with acetic acid, citric acid, lactic acid or tartaric acid, polyoxyethylene stearates, carbohydrate esters, propylene glycol esters, glycerol esters or sorbitan esters of edible fatty acids or sodium lauryl sulphate.

The present invention further relates to foods which contain the claimed formulations (functional foods). These can be, for example, drinks or bars which are particularly suitable for receiving the formulations. In a preferred embodiment, the food itself likewise does not contain any significant amounts of nitrogenous compounds, or is even free from nitrogenous compounds.

The claimed formulations can be added to the foods during their production, or a preparation of the food supplement can be added to the food later, for example in the form of a powder or a tablet. For example, here, the dissolution of effervescent tablets or of a powder can be initiated in mineral water.

The claimed formulations promote nitrogen detoxification or ammonia detoxification in muscles, which, inter alia, is necessary owing to the protein and amino acid breakdown in the muscles. Transfer of liberated amino groups to the keto acids generates the corresponding amino acids, and these are in turn available for muscle synthesis, and the energy-expensive nitrogen detoxification and excretion via liver and kidneys is decreased. Accordingly, fewer nitrogenous breakdown products, for example urea, are detected in the blood or urine. At the same time, the efficiency of the muscles is increased, or the muscle synthesis is supported by the food supplements, since by transamination, the administered keto acids in the muscle can be converted into the corresponding amino acids which are there available for anabolic reactions. Finally, a more rapid regeneration of the muscle tissue is established and the physical efficiency is improved.

Since ammonia accumulation can definitely have an effect on the central nervous system with increased stress or fatigue symptoms, this biological effect of keto acids can act on psychosomatic aspects, and so physical training can be carried out with more scope and at a higher intensity and with shorter regeneration time. This is of importance in particular for patients with Diabetes mellitus type II, since the disease pattern is frequently associated with lack of physical movement and reduced physical capacity, which, inter alia, can have ammonia accumulation as a cause. It has been found that via the potentially biological function of the keto acid, ammonia accumulation during physical training can be prevented or at least reduced, and so the patients can be more active and train more. With increased physical training, then improved glucose metabolism may also be expected.

From the abovementioned aspects, the formulation according to the invention and foods containing it are directed in particular towards diabetics who wish to treat the diabetes, in particular that of type II, in a supporting manner via sporting activity. The use of these products by elderly persons, who are known frequently to suffer additionally from restricted nitrogen transport or restricted nitrogen excretion capacity, is likewise particularly advantageous.

Therefore, the present invention further relates to the use of keto acids for producing orally consumable formulations and products such as, for example functional foods, tablets, powders, etc., for normalizing a diabetic metabolic state in diabetics, for muscle synthesis, for restricting the efficiency of the musculature, for protecting the musculature against cell damage under stress and for increasing the general feeling of wellbeing.

### Experimental Procedure

For determining the improvement in stamina, the individual anaerobic-aerobic threshold (IAAT) is determined. This proceeds on the basis of measuring a lactate-performance curve using a treadmill test (training phase protocol: start 6 km/h, increase 2 km/h, which corresponds to an increase of approximately 25-50 Watt/min, stage duration 3 min). Before and after a training stage, blood samples are taken in a 30-second pause and the glucose and lactate values determined by means of a YSI 2300 STAT plus analyzer from YSI Life Sciences, Yellow Springs, USA, and the maximum oxygen intake (VO₂ₘₐₓ) determined spirometrically using a K4 measuring instrument from Cosmed (Rome, Italy).

The improvement in jumping power can be measured using a jumping power measuring plate from Kistler, Winterthur, Switzerland. For determining the explosive force by means of the jumping power test, the protocols specific to the apparatus “squat jump” and “count movement jump” are used. The jumping power is measured on the basis of the contact time on the measuring plate and the jump height and is calculated in comparison with the body weight.

For determining the damage to muscle cells, for example during physical exertion, the uric acid level in the blood or urine, or the creatine kinase activity in the blood, is determined. The increase in creatine kinase activity correlates with the extent of muscle damage and can be determined by an enzymatic reaction using the Kit No. 1087533 from Roche Diagnostics, Mannheim, Germany. The uric acid level can be determined photometrically using the “Fluitest UA®” kit from Biocon Diagnostics, Vöhl/Marienhagen, Germany.

The effects of the claimed food supplement on protein metabolism may be demonstrated by determining urea in the blood or urine. The urea level can be determined using photometric end-point determination at a wavelength of 334 nm, using the urea S test combination (reagent kit No. 777510 from Boehringer Mannheim, Germany).

(Brunetti, A. and I. D. Goldfine. “Role of myogenin in myoblast differentiation and its regulation by fibroblast growth factor.” J. Biol. Chem. 265.11 (1990): 5960-63. Fernandez, A. M., et al. “Muscle-specific inactivation of the IGF-I receptor induces compensatory hyperplasia in skeletal muscle.” J. Clin. Invest 109.3 (2002): 347-55. Ragolia, L., Q. Zuo, and N. Begum. “Inhibition of myogenesis by depletion of the glycogen-associated regulatory subunit of protein phosphatase-1 in rat skeletal muscle cells.” J. Biol. Chem. 275.34 (2000): 26102-08. Sun, Z., et al. “Muscular response and adaptation to diabetes mellitus.” Front Biosci. 13 (2008): 4765-94.)

### EXAMPLES

### Study Procedure

In order to test the effect of a mixture of branched-chain alpha keto acids (BCKAs) and AKG in combination with physical training on glucose and insulin metabolism, muscle synthesis, increase in muscle efficiency, nitrogen metabolism and the improvement of general feeling of wellbeing in the case of Diabetes mellitus type II patients, we carried out the following human study:

### Subjects:

Two groups each of 15 subjects were recruited. These 30 subjects were evaluated in accordance with the inclusion criteria of the study plan and nominated on the basis of clinical and anthropometric data. These subjects were then randomized in a “double-blind” manner (Table 1). There was no statistically significant difference between the two groups with respect to sex distribution, age and body height.

TABLE 1
Anthropometric data of the subjects on recruitment

| | N | Sex | Age (years) | Height (cm) |
|---|---|---|---|---|
| Total | 30 | 7 f | 60 ± 10 | 173 ± 8 |
| Placebo | 15 | 3 | 60 ± 12 | 174 ± 7 |
| group | | | | |
| KAS group | 15 | 4 | 60 ± 9 | 171 ± 10 |

Mean ± standard deviation

| | N | Age (years) | Height (cm) |
|---|---|---|---|
| Total | 30 | 62 (51-70) | 175 (168-178) |
| Placebo | 15 | 61 (49-72) | 174 (168-180) |
| group | | | |
| KAS group | 15 | 63 (52-68) | 175 (166-178) |

Median with quartiles

### Training:

Physical training was carried out in two variants. One variant was carried out in the sport and rehabilitation section of the Ulm University Clinic under the care of sports scientists or graduate students, or in a fitness studio/physiotherapy practice under the supervision of a qualified trainer. The other variant was termed “free training”, supervised by the subjects themselves. The professionally supervised training counted as “training required for the study”, specifically three training units per week, and the free training as “additional training”. The training required for the study consisted of endurance training and strength-endurance training, wherein one training unit comprised endurance training of 15 minutes each repeated three times with intermediate pauses of about 5 minutes and strength-endurance training over 5 minutes. This resulted in a training time corresponding to the study plan for endurance with 45 minutes and the strength-endurance training with 5 minutes per training unit and therefore 135 minutes endurance training and 15 minutes of strength-endurance training per week. This training was carried out for 6 weeks. Then a regeneration phase of one week followed, in which no training was undertaken.

### 1.1 Keto Acid Supplementation

During the entire study phase of 7 weeks (6 training weeks and one regeneration week), the two subject groups consumed each day the amount matched to their body weight of Mix 2 (keto acids in the composition described below) or placebo mix, wherein one subject always consumed the same mix over the entire period. We selected the following composition of the food supplement:

### Keto Acids Per 500 mg Tablet:

| Keto acid blend (MIX 2) | Short cut | Amount | |
|---|---|---|---|
| Alpha-Ketoleucine Calcium | KIC-Ca | 95.22 | mg/Tablet |
| Alpha-Ketovaline Calcium | KIV-Ca | 60.36 | mg/Tablet |
| Alpha-Ketoisoleucine Calcium | KMV-Ca | 45.24 | mg/Tablet |
| Alpha-Ketoglutarate Sodium | AKG-Na | 199.18 | mg/Tablet |
| Total | | 400 | mg/Tablet |

### Aids Per 500 mg Tablet:

| Final blend | Chemical | Amount | |
|---|---|---|---|
| Keto acid or | | 400.0 | mg/Tablet |
| Placebo blend | | | |
| C*PharmGel 03415 | Maize starch | 10.0 | mg/Tablet |
| C*PharmGel 12012 | Maize starch | 20.0 | mg/Tablet |
| Aerosil ® 200 | Silicon dioxide | 2.5 | mg/Tablet |
| Avicel ® PH101 | Micro crystalline | 35.0 | mg/Tablet |
| | cellulose | | |
| Avicel ® PH200 | Micro crystalline | 20.0 | mg/Tablet |
| | cellulose | | |
| Kollidon ® 25 | Polyvinylpyrrolidone | 7.5 | mg/Tablet |
| Mg-stearic | | 5.0 | mg/Tablet |
| Total | | 500.0 | mg/Tablet |

### Coating Per 500 mg Tablet:

| Formulation | Amount | |
|---|---|---|
| EUDRAGIT ® | 4 | Mg/cm² |
| Talc | 50 | % based on polymer |
| Stearic acid | 15 | % based on polymer |
| Sodium lauryl | 10 | % based on polymer |
| sulphate | | |
| Candurin ® Orange | 10 | % based on polymer |
| Amber | | |
| Water | 85% | % based on total amount of |
| | | coating suspension |
| | | % based on total amount of |
| | | coating suspension |
| Solid content | 15% | |

Eudragit® EPO is a methacrylate copolymer (Pharma Polymere, No. 9, November 2002, pp. 1-4). This agent masks odour and flavour.

### Composition of Placebo Tablet in mg Per 500 mg Tablet:

| | |
|---|---|
| CaHPO₄ | 41.6807625 |
| NaHCO₃ | 42.02211054 |
| Fructose | 166.297127 |

in total 250 mg of “placebo active ingredient”

in addition, 250 mg of aids are added:

| | |
|---|---|
| C Gel LM 03411 | 6.25 mg |
| C Pharma Gel 12012 | 12.5 mg |
| Avicel PH101 | 141.2 mg |
| Avicel PH200 | 80.7 mg |
| Kollidon 25 | 7.8 mg |
| Magnesium stearate | 1.6 mg |

| | Weight fraction in % in the end |
|---|---|
| Substance | product |
| Fructose | 33.30 |
| Sodium hydrogencarbonate | 8.40 |
| Calcium hydrogenphosphate | 8.30 |
| C*Gel ® LM 03411 | 1.25 |
| C*PharmGel ® 12012 | 2.5 |
| Avicel ® PH 101 | 28.24 |
| Avicel ® PH 200 | 16.14 |
| Kollidon ® 25 | 1.56 |
| Magnesium stearate | 0.31 |
| TOTAL | 100.00 |

Each subject consumed per day 0.2 g of the keto acid group/kg of body weight/day of the said mixture. In the study, AKG was administered as sodium salt and KIC, KIV and KMV as calcium salts. The subjects of the placebo group consumed the same amount of energy and salts. They consumed 1.45 placebo tablets per kg of body weight/day.

### 1.1.1 Effect on Maximum Physical Performance

In FIG. 1, the maximally achieved physical performance in the ramp test is summarized. The maximally achieved physical performance before the start of training appeared to be somewhat higher in the KAS group than in the placebo group, which statistically, however, did not differ significantly (P>0.05). Overall, a marked increase of this maximum performance due to physical training was demonstrated during the study period. In all subjects the maximally achieved physical performance recorded a marked increase after the training programme and also after the regeneration (P<0.01 and P<0.05, respectively).

Training lead to an increase in physical performance both in the placebo group and in the KAS group. However, the performance increase in the KAS groups was higher and remained for longer.

The physical training can be improved by the higher performance.

### 1.1.2 Effect on Stamina

For stamina, the physical performance determined in the multistep test at the individual aerobic-anaerobic lactate threshold was used for the evaluation. However, this parameter could not always be determined in the case of relatively physically weak subjects, and so they varied additionally (Table 2).

TABLE 2
Performance at individual aerobic-anaerobic threshold
(watts, mean ± standard deviation)

| | | Time point | | |
|---|---|---|---|---|
| | n | 1 | 2 | 3 |
| Total | 26 | 88.4 ± 30.3 | 101.8 ± 35.9 | 100.7 ± 34.2 |
| Group 0 | 12 | 86.0 ± 37.8 | 95.9 ± 42.3 | 96.4 ± 40.8* |
| Group 1 | 14 | 90.4 ± 23.6 | 108.3 ± 28.3* | 103.2 ± 31.1* |

The result shows that the physical performance is markedly increased by the physical training for the subjects overall (FIG. 2).

The performance increase of the KAS groups was greater than that of the placebo group.

### 1.1.3 Effect on Glucose Metabolism

In FIG. 3, the result for glucose concentration in plasma is shown.

The glucose level in the blood is considered to be a control parameter for glucose metabolism in diabetics. In the present study, this level was established to be relatively good even before the start of the study. In the KAS group a slightly poorer level was established.

Overall, the glucose level before training was slightly elevated, wherein it was higher in the KAS group than in the placebo group, although this difference was not statistically significant.

It was found that the glucose level was markedly reduced by the physical training by 16 mg/dl in the placebo group and 11.5 mg/dl in the KAS group. After one week of regeneration the glucose level in the placebo group increased again slightly (P<0.05), but decreased further in the KAS group (although P>0.05). After the 7 weeks of intervention, in the placebo group a decrease by 9 mg/ml was found, and in the keto acid group in contrast by greater than 20 mg/ml!

In the placebo group, training caused a significant decrease of the glucose level in blood, such that it was in the physiological range (FIG. 3) and still remained below the starting level after the regeneration phase. This result clearly shows, as widely described in the literature, that physical training has a beneficial effect on glucose metabolism in diabetics. However, the beneficial effect of physical training on glucose metabolism does not appear to last long, and so the glucose level in blood significantly increased again. This implies that physical training for diabetics should be a therapeutic measure rather than a “long-lasting therapy”.

In the KAS group, the glucose level in the regeneration phase decreased further, and so the glucose level at the end of the study period still remained significantly below the starting level. This result shows in comparison to the placebo group:

1). A greater decrease of the glucose level in the blood, since the starting value in the KAS group was higher (pathological);
2). The glucose-lowering effect of physical training was retained longer by KAS. In particular, the further decrease of the glucose level in the regeneration phase indicates an improved insulin function, since in this phase scarcely any training was carried out.

### 1.2 HbA1c

A long-term parameter of glucose metabolism is HbA1c (FIG. 4). In the subjects, the HbA1c fraction was somewhat elevated at the start of the study, but more markedly in the KAS group. As a result of the training, this decreased significantly to the virtually normal level in both groups. Therefore, the “net gain” in the lowering of the HbA1c in the KAS group was markedly higher than that in the placebo group, which argues for a greater effect.

In summary, it may be stated that the physical training has led to a marked improvement of glucose metabolism in diabetics. KAS acts additionally greater on the glucose control and has a longer-lasting effect.

### 1.3 Quantitative Insulin Sensitivity Check Index (Quicki)

QUICKI (quantitative insulin sensitivity check index) is a widespread parameter of insulin sensitivity and is based on the basal insulin level and the glucose level. An increasing QUICKI indicates an improved insulin sensitivity. That means, the lower the insulin level is for a defined glucose level, the higher is the insulin sensitivity.

This value is calculated according to the formula:

QUICKI=1/[log(basal insulin [u/L]+log(glucose [mg/dl])]

A description of this method may be found in

### Wallace T M, Levy J C and Matthew D R. Use and Abuse of HOMA Modeling, Diabetes Care 27: 1487-1495, 2004.

FIG. 5 shows that QUICKI was still unchanged in the placebo group after training and only increased after the regeneration phase, and the changes were not statistically significant. This means that the insulin sensitivity in the placebo group after training remained unchanged and increased at the end of the study period (but not statistically significantly). In the KAS group, QUICKI behaved differently than in the placebo group. There was a significant increase after training and a reduction in the regeneration phase, but above the starting level. The significant increase in the QUICKI value in the KAS group therefore indicates an improved insulin sensitivity.

### EXPLANATION OF THE FIGURES

FIG. 1: Maximally achieved physical performance in the ramp test during the study period in the placebo group (placebo) and the group with keto acid supplementation.

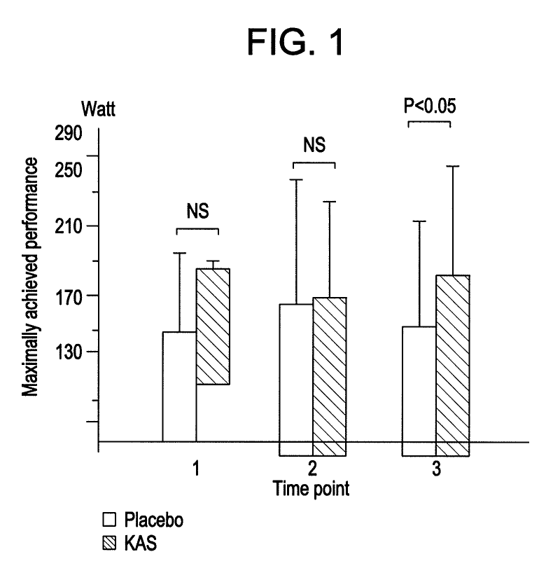

FIG. 2: Physical performance at individual aerobic-anaerobic lactate threshold in the multistep test during the study period in the placebo group (placebo) and the group with keto acid supplementation.

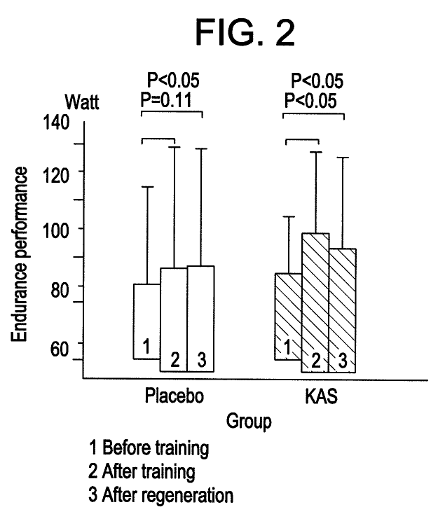

FIG. 3: Glucose level in plasma during the study period in the placebo group (placebo) and the group with keto acid supplementation (mean±standard deviation).

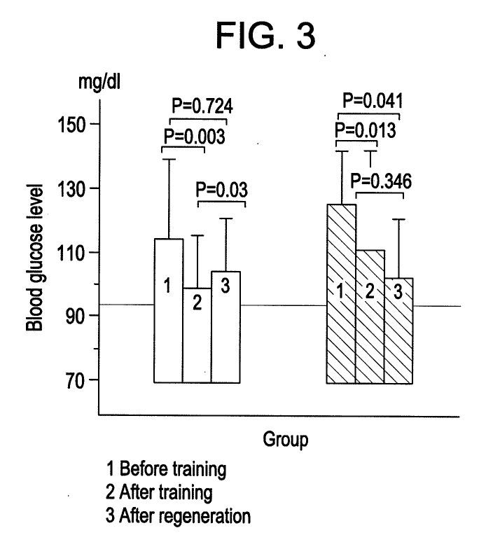

FIG. 4: HbA1c in the plasma during the study period in the placebo group (placebo) and the group with keto acid supplementation.

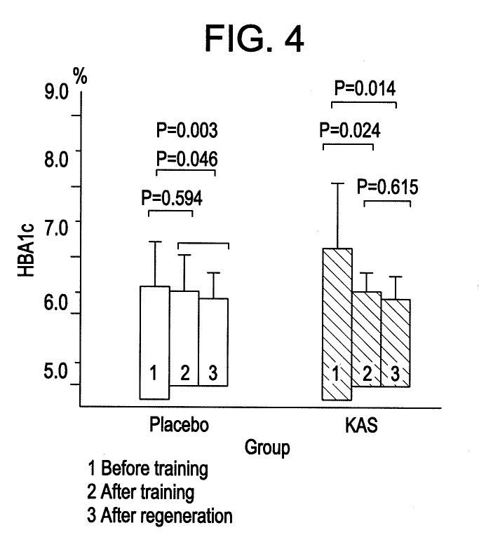

FIG. 5: Quantitative insulin sensitivity check index during the study period in the placebo group (placebo) and the group with keto acid supplementation (median).

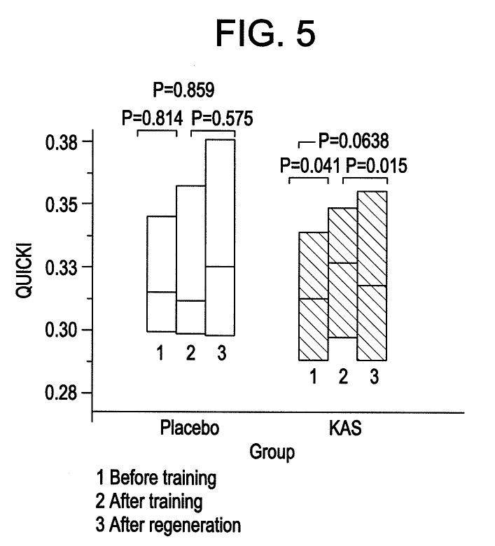

1. A formulation which contains one or more alpha-keto acids and/or salts thereof selected from the group alpha-ketoglutarate, alpha-ketoisocaproate, alpha-ketoisovalerate and alpha-keto-beta-methylvalerate and/or salts thereof, wherein the formulation is a food supplement and is essentially nitrogen-free.

2. The formulation according to claim 1, in which the alkali metal or alkaline earth metal salts, in particular the Na⁺, K⁺, Ca²⁺ and Mg²⁺ salts of the said alpha-keto acids are contained.

3. The formulation according to claim 1, which contains keto acids in the quantitative ratio of AKG/BCKAs from 5:1 to 1:5, particularly preferably 2:1 to 1:2.

4. The formulation according to claim 1, which contains a daily dose of total amount of alpha-keto acids between 0.5 g and 50 g, particularly preferably between 1.25 g and 25 g.

5. The formulation according to claim 1, which additionally contains L-ornithine, L-lysine, L-histidine or L-arginine, wherein the total nitrogen content of the formulation is <6% by weight.

6. The formulation according to claim 5, which contains the amino acids as salts of the said alpha-keto acids.

7. The formulation according to claim 1 which additionally contains creatine.

8. A food supplement which comprises a formulation according to claim 1, wherein the food supplement contains further additives selected from the group of carbohydrates, fats and oils, vitamins, antioxidants, minerals and trace elements, preservatives, food dyes, sweeteners, taste enhancers and flavourings.

9. The food supplement according to claim 8 and further comprising formulation aids.

10. The food supplement according to claim 8 and further comprising a methacrylate copolymer, in particular Eudragit® E PO.

11. A food containing the food supplement according claim 8.

12. A method of producing orally consumable products for supporting a diabetes therapy, increasing the efficiency of the musculature, for protecting the musculature from cell and tissue damage, for increasing the general physical efficiency and/or for supporting muscle regeneration after physical stress with simultaneous relief of metabolism with respect to nitrogen detoxification which comprises using the formulation according to claim 1.

13. A method for supporting muscle synthesis during physical training in a subject which comprises administering to the subject a formulation according to claim 1.

14. The method of claim 13, wherein the subject has Diabetes mellitus type II.

15. A method of normalizing a diabetic metabolic state and a reduction of Hbc1a in a subject which comprises administering the formulation of claim 1 to the subject in combination with physical activity.

16. A method of lowering a blood glucose level in a subject which comprises administering the formulation of claim 1 to the subject in combination with physical activity.

17. A method of increasing insulin sensitivity in a subject which comprises administering the formulation of claim 1 to the subject.

18. A method of treating diabetes in a mammalian subject which comprises administering to the subject the formulation of claim 1.
